(19) United States
(12) Patent Application Publication
(10) Pub. No.: US 2014/0080160 A1
(43) Pub. Date: Mar. 20, 2014

(54) Assessment of Protein C Anticoagulant Pathway By Thrombin Generation Assay In The Presence of Endothelial Cells

(71) Applicant: Adventist Health System/Sunbelt, Inc., Altamonte Springs, FL (US)

(72) Inventors: Enriqueta Coll, Orlando, FL (US); Liza Robles-Carrillo, Rockledge, FL (US); John Francis, Longwood, FL (US); Ali Amirkosravi, Longwood, FL (US)

(73) Assignee: ADVENTIST HEALTH SYSTEM/SUNBELT, INC., Altamonte Springs, FL (US)

(21) Appl. No.: 13/964,148

(22) Filed: Aug. 12, 2013

Related U.S. Application Data
(60) Provisional application No. 61/681,691, filed on Aug. 10, 2012.

(51) Int. Cl. C12Q 1/56

(52) U.S. Cl. CPC C12Q 1/56
USPC 435/13

(57) ABSTRACT

The present application discloses the effects of endothelial cell-dependent activation of the protein C anticoagulant pathway via detection of thrombin generation correlated with the activation of the protein C anticoagulant pathway in plasma in the presence of endothelial cells using a fluorogenic substrate-based thrombin generation assay.

### CROSS-REFERENCE TO RELATED APPLICATIONS

This application is entitled to the benefit of U.S. provisional patent application Ser. No. 61/681,691 filed on Aug. 10, 2012, entitled “Assessment of Protein C Anticoagulant Pathway By Thrombin generation Assay In The Presence of Endothelial Cells” the disclosure of which is incorporated herein by reference.

### FIELD OF THE INVENTION

The present invention generally relates to thrombin generation detection and in particular to an assessment of protein C anticoagulant pathway using thrombin generation assay in the presence of endothelial cells.

### BACKGROUND

The generation of thrombin is the pivotal event in the process of blood coagulation. In vivo, thrombin generation is regulated by cooperation between the vascular endothelium and the pro- and anti-coagulant systems in blood, such as the thrombin/endothelial cell-dependent activation of the protein C anticoagulant pathway that ultimately leads to factors Va and VIIIa inactivation.

In vitro, thrombin generation is assessed most commonly in plasma by a fluorogenic substrate-based assay (TGA) in microtiter plates. While this assay can accurately measure the kinetics of thrombin generation in plasma (including lag time [LT], peak thrombin [PT], and endogenous thrombin potential [ETP]), it does not assess the influence of the endothelial cell-dependent protein C pathway on thrombin generation. As a result, the assay has limited value in the assessment of the hypercoagulable patient. Therefore, in order to make the assay sensitive to the inactivation of factors Va and VIIIa by the activated protein C (APC) system in a physiologically relevant manner a surrogate endothelial component to the TGA , was introduced.

### SUMMARY OF THE INVENTION

By introducing an endothelial cell monolayer to the TGA, and measuring thrombin generation kinetics in the presence or absence of cells, an assay may be adapted assess to the contribution of the protein C anticoagulant system to thrombin generation in a physiologically relevant manner. According to the teachings of the present invention, this approach not only enables the functioning of the endothelial-dependent PC pathway by expressing TM and EPCR, but also may provide other endothelial components relevant to thrombin generation (such as tissue factor pathway inhibitor [TFPI]) in the assessment of thrombin generation. This approach to thrombin generation assessment may therefore have both research and clinical applicability.

The application provides a method of determining plasma thrombin levels comprising:
a) providing plasma in the presence of endothelial cell-dependent activation of the protein C anticoagulant pathway; and
b) subjecting said plasma to a fluorogenic substrate-based thrombin generation assay.

The application provides the above method, wherein the endothelial cell-dependent activation of the protein C anticoagulant pathway is provided by introduction of a surrogate endothelial component.

The application provides the above method, wherein the surrogate endothelial component comprises quiescent EA.hy926 cells.

The application provides the above method, wherein the surrogate endothelial component is a monolayer.

The application provides a method of assessing the influence of the protein C anticoagulant pathway on thrombin generation comprising:
a) providing plasma in the presence of endothelial cell-dependent activation of the protein C anticoagulant pathway; and
b) subjecting said plasma to a fluorogenic substrate-based thrombin generation assay.

The application provides the above method, wherein the endothelial cell-dependent activation of the protein C anticoagulant pathway is provided by introduction of a surrogate endothelial component.

The application provides the above method, wherein the surrogate endothelial component comprises quiescent EA.hy926 cells.

The application provides a method of testing plasma for inflammatory conditions comprising:
a) providing plasma in the presence of endothelial cell-dependent activation of the protein C anticoagulant pathway; and
b) subjecting said plasma to a fluorogenic substrate-based thrombin generation assay.

The application provides the above method, wherein the endothelial cell-dependent activation of the protein C anticoagulant pathway is provided by introduction of a surrogate endothelial component.

The application provides the above method, wherein the surrogate endothelial component comprises quiescent EA.hy926 cells.

The application provides a method of assessing the contribution of endothelial cell-dependent activation of the protein C anticoagulant pathway to the generation of thrombin under normal or pathological conditions comprising:
a) providing plasma in the presence of protein C anticoagulant pathway; and
b) subjecting said plasma to a fluorogenic substrate-based thrombin generation assay.

The application provides the above method, wherein the endothelial cell-dependent activation of the protein C anticoagulant pathway is provided by introduction of a surrogate endothelial component.

The application provides the above method, wherein the surrogate endothelial component comprises quiescent EA.hy926 cells.

The application provides a method of assessing thrombin generation in plasma under hypercoagulable conditions comprising:
a) providing plasma in the presence of endothelial cell-dependent activation of the protein C anticoagulant pathway; and
b) subjecting said plasma to a fluorogenic substrate-based thrombin generation assay.

The application provides the above method, wherein the endothelial cell-dependent activation of the protein C anticoagulant pathway is provided by introduction of a surrogate endothelial component.

The application provides the above method, wherein the surrogate endothelial component comprises quiescent EA.hy926 cells.

The application provides a method of assessing the effect of endothelial cell-dependent activation of the protein C anticoagulant pathway mediated by TM, EPCR, and/or TFPI on thrombin generation comprising:
a) providing plasma in the presence of endothelial cell-dependent activation of the protein C anticoagulant pathway; and
b) subjecting said plasma to a fluorogenic substrate-based thrombin generation assay.

The application provides the above method, wherein the endothelial cell-dependent protein C pathway is provided by introduction of a surrogate endothelial component.

The application provides the above method, wherein the surrogate endothelial component comprises quiescent EA.hy926 cells.

The application provides a method of assessing the effect of endothelial cell-dependent activation of the protein C anticoagulant pathway inactivation of factors Va and VIIIa on thrombin generation comprising:
a) providing plasma in the presence of endothelial cell-dependent activation of the protein C anticoagulant pathway; and
b) subjecting said plasma to a fluorogenic substrate-based thrombin generation assay.

The application provides the above method, wherein the endothelial cell-dependent activation of the protein C anticoagulant pathway is provided by introduction of a surrogate endothelial component.

The application provides the above method, wherein the surrogate endothelial component comprises quiescent EA.hy926 cells.

The application provides any of the above methods in the assessment of a thrombin mediated condition or disease.

The application provides any of the above methods in the assessment of a protein C anticoagulant pathway mediated condition or disease.

### BRIEF DESCRIPTION OF THE DRAWINGS

Embodiments of the invention are described by way of example with reference to the accompanying drawing in which:

FIG. 1A provides thrombogram profiles obtained in the presence and absence of endothelial cells for normal plasma.

FIG. 1B provides thrombogram profiles obtained in the presence and absence of endothelial cells for protein C deficient plasma.

FIG. 1C provides thrombogram profiles obtained in the presence and absence of endothelial cells for protein S deficient plasma.

FIG. 1D provides thrombogram profiles obtained in the presence and absence of endothelial cells factor V Leiden (D) plasma.

FIGS. 1E and 1H exhibit the dose-dependent reversed blunted inhibition of thrombin generation observed in PCd when mixed with NP to obtain different percentages of abnormal plasma.

FIGS. 1F and 11 exhibit the dose-dependent reversed blunted inhibition of thrombin generation observed in PSd when mixed with NP to obtain different percentages of abnormal plasma.

FIGS. 1G and 1J exhibit the dose-dependent reversed blunted inhibition of thrombin generation observed in fVL when mixed with NP to obtain different percentages of abnormal plasma.

FIG. 1K exhibits the effect of incubation of NP with anti-TFPI in the absence of cells, and in the presence of EA.hy926 cells.

FIGS. 1L and 1M exhibit the sensitivity of the TGA to addition of EA.hy926 cells on plasma levels of PC, PS and fVL; as well as corn-trypsin inhibitor.

### DETAILED DESCRIPTION OF THE PREFERRED EMBODIMENTS

The present invention will now be described more fully hereinafter with reference to the accompanying drawing, in which embodiments of the invention are described by way of example. This invention may, however, be embodied in many forms and should not be construed as limited to the embodiments set forth herein. Rather, these embodiments are provided so that this disclosure will be thorough and complete, and will fully convey the scope of the invention to those skilled in the art.

According to the teachings of the present invention, a surrogate endothelium was introduced to the TGA, thereby providing activated protein C-induced inactivation of factors Va and VIIIa in the assay system.

### Experimental

### EXAMPLE 1

Wells of flat-bottomed microtiter plates were coated with quiescent EA.hy926 cells (hybrid of human umbilical vein endothelial cells [HUVEC] and A549 lung carcinoma cells; ATCC, Manassas, Va., USA) which consistently express thrombomodulin (TM) as well as the endothelial protein C receptor (EPCR). This cell line was chosen to provide endothelial cell-like APC function without the reproducibility and stability problems associated with endothelial cells from natural sources (e.g. HUVEC). EA.hy926 cells were cultured to 80% confluence in 75 cm² flasks in Dulbecco's Modified Eagle Medium containing 10% fetal bovine serum. In order to establish quiescensce, cells were washed in PBS and further incubated in serum-free medium (SFM) for 24 hours. Cells were then washed, harvested and seeded (4×10⁴ cells in 200 μL of complete growth medium) in half of the wells (48 wells) of a tissue culture grade 96-well microtiter plate (Costar 3599, Corning) and incubated for 12-18 hours to allow optimum attachment, while avoiding further cell proliferation. Finally, EA.hy926 monolayers were washed and incubated for 24 hours in SFM to re-establish quiescence. Before assay, monolayers were washed once with PBS.

Thrombin generation was evaluated according to the method by Hemker et al in normal pooled plasma (NP; Precision Biologics, Dartmouth, Canada), and in protein C-deficient (PCd; Affinity Biologicals, Ancaster, Canada), protein S-deficient (PSd; Affinity Biologicals), and heterozygous factor V Leiden (fVL; from individual subjects) plasmas, in the presence or absence of EA.hy926 monolayers. After addition of 5 μM (final concentration [f.c.]) recombinant relipidated human tissue factor (TF; Innovin, Dade Behring, Newark, USA) and 15 μM (f.c.) phospholipids (PL; DOPS/DOPC/DOPE, Avanti Polar Lipids, Alabaster, USA) to plasmas, thrombin generation was initiated by a mixture of 16.7 mM (f.c.) CaCl₂ and the fluorogenic substrate 1-1140 (Z-Gly-Gly-Arg-AMC.HCl, 417 μM [f.c.]; Bachem, Torrance, USA), and monitored for 90 minutes. Assay calibrator was run for each sample (with or without cells) by adding alpha 2-macroglobulin-thrombin complex (Diagnostica Stago, Parsippany, USA) instead of TF/PL.

By using a chromogenic assay for measuring APC activity, we found that membrane-bound TM from 4×10⁴ intact EA.hy926 cells (per well) had an activity equivalent to ˜0.5 nM of soluble thrombomodulin (sTM; Haematologic Technologies, Essex Junction, USA).

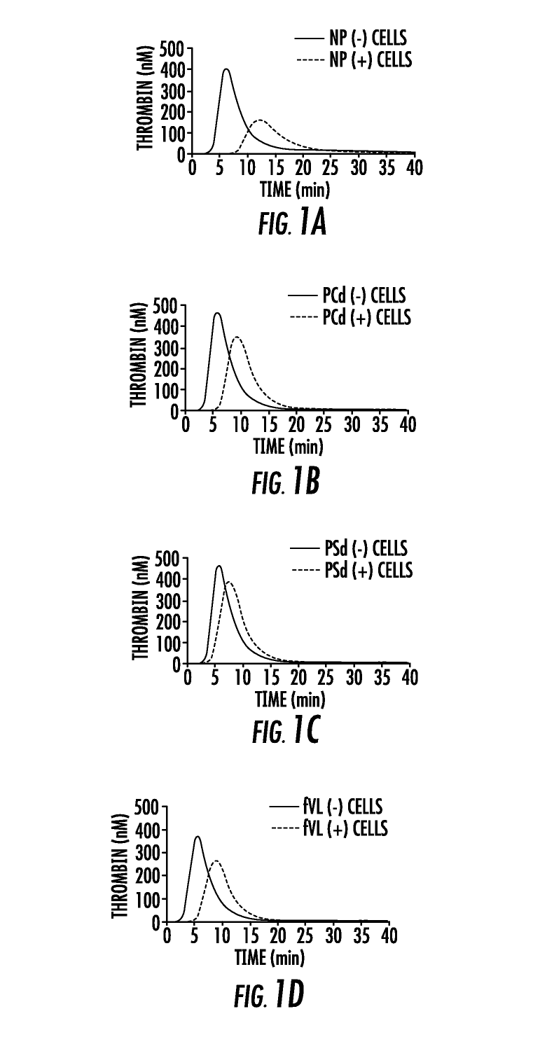

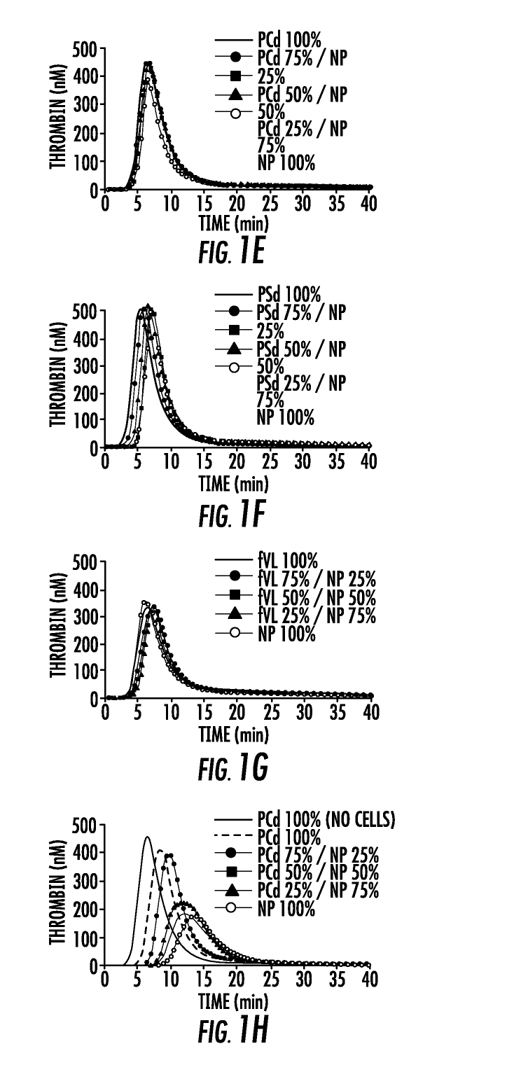

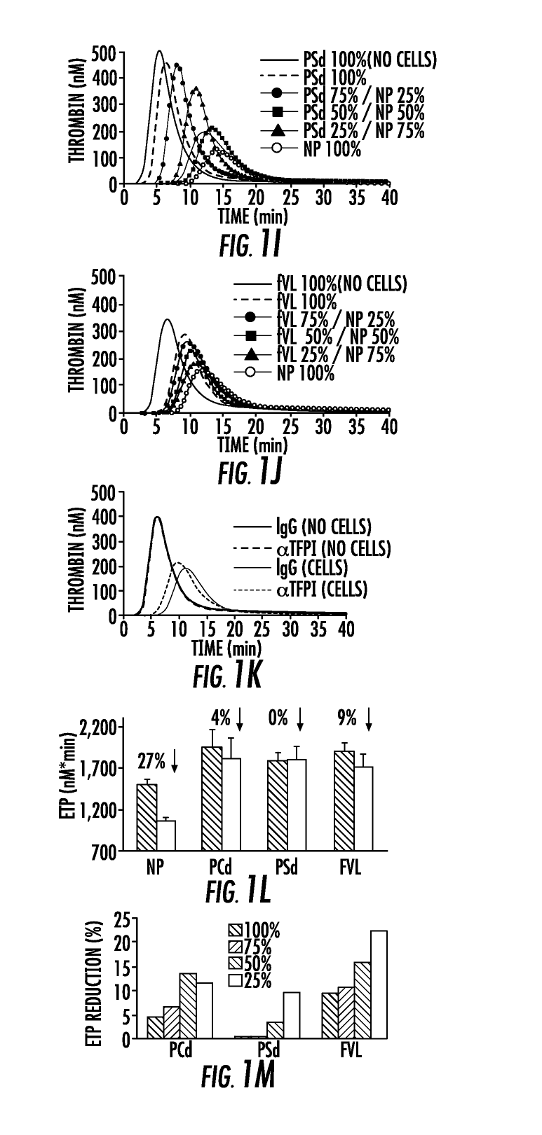

When compared with bare microtiter wells, thrombin generation in NP was suppressed by EA.hy926 cells as evidenced by a 56% reduction in PT, 39% reduction in ETP and 107% prolongation of LT (FIG. 1A). However, in PCd, PSd and fVL plasmas, endothelial-induced suppression of thrombin generation was blunted (FIGS. 1B, C and D, respectively). Specifically, smaller reductions were observed in PT (31% in PCd, 13% in PSd, 23% in fVL) and ETP (11% in PCd, 7% in PSd, and 18% in fVL). In addition, compared to NP, the prolongation of LT in the presence of cells was less pronounced in PCd (68%), PSd (30%) and fVL (61%) plasmas.

### EXAMPLE 2

Furthermore, the blunted inhibition of thrombin generation observed in PCd, PSd and fVL plasmas was dose-dependently reversed when these plasmas were mixed with NP to obtain different percentages of abnormal plasma. This was evident by prolongation of LT and reductions in PT and ETP (FIGS. 1E and H for PCd; FIGS. 1F and 11 for PSd; FIGS. 1G and 1J for fVL). Similar results were obtained when PCd and PSd plasmas were spiked with purified protein C and S at different concentrations (not shown).

Together, these results show that the addition of EA.hy926 cells to the TGA makes the assay sensitive to plasma PC and PS levels as well as the presence of fVL. When this assay was performed in the presence of corn-trypsin inhibitor to ensure exclusive TF-dependent thrombin generation, similar results were obtained, even though in general (as expected) less thrombin was generated (FIGS. 1L and 1M).

### EXAMPLE 3

In separate experiments in the absence of cells, we found that a sixty-fold increased quantity of the soluble form of TM (30 nM) was required to inhibit thrombin generation to the same degree as the EA.hy926 monolayer (not shown), indicating that protein C activation occurs more efficiently in the presence of EA.hy926 than with sTM. This difference could be attributed to the presence of cell surface EPCR which enhances PC activation by the thrombin-TM complex by ˜20 fold.

### EXAMPLE 4

EA.hy926 cells also express tissue factor pathway inhibitor (TFPI). To assess the potential contribution of TFPI to the anticoagulant effects of the monolayer under our experimental conditions, we measured thrombin generation in the presence of blocking anti-TFPI antibody with or without EA.hy926. After incubation of NP with anti-TFPI (50 μg/ml; Cell Sciences, Canton, USA) in the absence of cells, thrombin generation was only marginally affected, as evidenced by a 7% increase in ETP while PT and LT were unchanged. In the presence of EA.hy926, thrombin generation was slightly increased by anti-TFPI (ETP increase of 18%, PT increase of 22% and LT shortening of 18%; FIG. 1K), suggesting that under our experimental conditions, the TFPI expressed by EA.hy926 cells had a relatively minor contribution to the overall cell-induced inhibition of thrombin generation. This was expected since, at the concentrations of TF used in this system, the large amounts of FXa formed are beyond regulation by the protein S-TFPI anticoagulant pathway.

Blocking anti-TFPI antibodies or aptamers have been previously used to evaluate the effect of TFPI on thrombin generation. The contribution of the EA.hy926 cells to whole blood coagulation in vitro was also shown by Campbell. In their study, where EA.hy926 cell-coated tubes were used as a source of endothelium in a TF-activated blood clotting assay (5 μM TF) in the presence of CTI, the contribution of EA.hy926-expressed TFPI to the reduction in thrombin generation was said to be negligible because of the similar coagulation initiation times in both, endothelial-coated and non-coated tubes. In our study, however, the LT of thrombin generation in normal platelet-free plasma (without CTI) was prolonged in the presence of EA.hy926 (FIG. 1A).

### Experimental Results Depicted in the Figures

FIGS. 1A-1D. Thrombin generation in the absence or presence of EA.hy926 monolayers. Thrombograms from normal (NP; 1A), protein C deficient (PCd; 1B), protein S deficient (PSd; 1C) and heterozygous factor V leiden (fVL; 1D) plasmas in the absence (solid line) or presence (dashed line) of EA.hy926 cells. Peak thrombin was decreased by 55.8±4.9% (371±34 nM without cells [−] vs. 170±13 nM with cells [+]) in NP and by 30.5±6.6% (464±18 nM [−] vs. 322±25 nM [+]) in PCd, 12.7±5.4% (473±16 nM [−] vs. 413±35 nM [+]) in PSd and 22.6±6.5% (369±21 nM [−] vs. 290±39 nM [+]) in fVL plasmas, respectively. ETP decreased 39.2±6.9% (2078±268 nM*min [−] vs. 1202±113 nM*min [+]) in NP, 10.5±8.3% (2293±66 nM*min [−] vs. 2054±213 nM*min [+]) in PCd plasma, 6.7±3.6% (2371±113 nM*min [−] vs. 2211±88 nM*min [+]) in PSd plasma and 18.2±6.1% (1889±140 nM*min [−] vs. 1571±161 nM*min [+]) in fVL plasma. Lag time was prolonged by 107±43.3% (3.4±0.3 min [−] vs. 6.9±1.1 min [+]), 68±2.7% (3.2±0.3 min [−] vs. 5.3±0.6 min [+]), 30±20% (2.6±0.2 min [−] vs. 3.4±0.3 min [+]), and 61±9.6% (3.1±0.3 min [−] vs. 4.8±0.3 min [+]), in NP, PCd, PSd and fVL plasmas, respectively. The thrombograms represent the results obtained from testing 20 NP and 9 for each of PCd, PSd, fVL plasmas. The intra-assay coefficients of variation (CV) of all thrombin generation parameters obtained from 10 replicates of normal plasma, showed good reproducibility as follows: In the absence of EA.hy926 cells, lag time: CV=7.4%; peak thrombin=2.7%; ETP=6.4%; in the presence of cells, lag time: CV=10.2%; peak thrombin=CV 9.5%; ETP=4.4%.

FIGS. 1E-1J. Thrombin generation in mixed plasmas (PCd, PSd and heterozygous fVL mixed with normal plasma) in the absence (1E, 1F, 1G) or presence (1H, 1I, 1J) of endothelial cells. PCd, PSd and fVL were mixed with normal plasma in order to obtain 75%, 50% and 25% of factor deficient or fVL plasmas. Thrombin generation assay was performed in these mixes as well as in the original plasmas in the presence or absence of EA.hy926 cells. The figures showing the results in the presence of cells also include the thrombograms of each plasma (PCd, PSd and fVL) without cells (100% [no cells]) in order to illustrate the inhibition of thrombin generation in both conditions. The thrombograms represent the results of 3 experiments.

FIG. 1K. Thrombin generation in the absence or presence of anti-TFPI antibody (aTFPI). NP and endothelial cells were incubated (20 minutes, 37° C.) with aTFPI (50 μg/ml f.c.) or control IgG. Thrombin generation was then measured in the absence or presence of endothelial monolayers. The thrombograms represent the results of 3 experiments. The TGA parameters were as follows: in the absence of endothelial monolayers, ETP (nM*min): (2113±171 [IgG] vs. 2254±66 [aTFPI]). PT (nM): (403±21 [IgG] vs. 408±14 [aTFPI]). LT (min): (3.0±0 [IgG] vs. 3.0±0.4 [aTFPI]). In the presence of endothelial monolayers: ETP: (1355±123 [IgG] vs. 1604±52 [aTFPI). PT: (186±21 [IgG] vs. 227±3 [aTFPI]. LT: (6.5±1.0 [IgG] vs. 5.3±0.6 [aTFPI].

FIG. 1 L. Thrombin generation with or without EA.hy926 cells in the presence of CTI. Bars chart shows ETP results obtained from NP (n=6), PCd (n=3), PSd (n=4), and fVL (n=4) plasmas in the absence (black bars) or presence (white bars) of EA.hy926 cells. ETP values in NP: 1494±120 nM*min [− cells] vs. 1096±205 nM*min [+ cells]); in PCd: 1917±188 nM*min [−cells] vs. 1833±204 nM*min [+cells]; in PSd: 1735±138 nM*min [− cells] vs. 1742±185 nM*min [+ cells]; and in fVL: 1894±108 nM*min [−cells] vs. 1715±157 nM*min [+cells].

FIG. 1 M. Thrombin generation inhibition by endothelial cells in mixed plasmas (PCd, PSd and heterozygous fVL mixed with normal plasma) in the presence of CTI. PCd, PSd and fVL were mixed with normal plasma in order to obtain 75%, 50% and 25% of factor deficient or fVL plasmas. Thrombin generation assay was performed in these mixes as well as in the original plasmas in the presence or absence of EA.hy926 cells. Bars chart shows the percentage of reduction of ETP in the original sample and mixes (average of 2-4 experiments).

In conclusion, this novel approach of TGA enables the functions of the endothelial cell-dependent protein C pathway mediated by TM and EPCR. The introduction of an endothelial component to the TGA may open the possibility for examining its contribution to thrombin generation under normal or pathological (e.g., inflammatory) conditions. Thus, this assay may have potential research applicability, although its clinical applicability may be limited, particularly in high throughput systems.

Although the invention has been described relative to various selected embodiments herein presented by way of example, there are numerous variations and modifications that will be readily apparent to those skilled in the art in light of the above teachings. It is therefore to be understood that, within the scope of the claims supported by this disclosure, the invention may be practiced other than as specifically described.

What is claimed is:

1. A method of determining plasma thrombin levels comprising:
a) providing plasma in the presence of endothelial cell-dependent activation of the protein C anticoagulant pathway; and
b) subjecting said plasma to a fluorogenic substrate-based thrombin generation assay.

2. The method of claim 1, wherein the endothelial cell-dependent activation of the protein C anticoagulant pathway is provided by introduction of a surrogate endothelial component.

3. The method of claim 2, wherein the surrogate endothelial component comprises quiescent EA.hy926 cells.

4. The method of claim 3, wherein the surrogate endothelial component is a monolayer.

5. A method of assessing the influence of the protein C anticoagulant pathway on thrombin generation comprising:
a) providing plasma in the presence of endothelial cell-dependent activation of the protein C anticoagulant pathway; and
b) subjecting said plasma to a fluorogenic substrate-based thrombin generation assay.

6. The method of claim 5, wherein the endothelial cell-dependent activation of the protein C anticoagulant pathway is provided by introduction of a surrogate endothelial component.

7. The method of claim 6, wherein the surrogate endothelial component comprises quiescent EA.hy926 cells.

8. A method of testing plasma for a protein C anticoagulant pathway mediated condition or disease comprising:
a) providing plasma in the presence of endothelial cell-dependent activation of the protein C anticoagulant pathway; and
b) subjecting said plasma to a fluorogenic substrate-based thrombin generation assay.

9. The method of claim 8, wherein the endothelial cell-dependent activation of the protein C anticoagulant pathway is provided by introduction of a surrogate endothelial component.

10. The method of claim 9, wherein the surrogate endothelial component comprises quiescent EA.hy926 cells.

11. A method of assessing the contribution of endothelial cell-dependent activation of the protein C anticoagulant pathway to the generation of thrombin under normal or pathological conditions comprising:
a) providing plasma in the presence of protein C anticoagulant pathway; and
b) subjecting said plasma to a fluorogenic substrate-based thrombin generation assay.

12. The method of claim 11, wherein the endothelial cell-dependent activation of the protein C anticoagulant pathway is provided by introduction of a surrogate endothelial component.

13. The method of claim 12, wherein the surrogate endothelial component comprises quiescent EA.hy926 cells.

14. A method of assessing thrombin generation in plasma under hypercoagulable conditions comprising:
a) providing plasma in the presence of endothelial cell-dependent activation of the protein C anticoagulant pathway; and
b) subjecting said plasma to a fluorogenic substrate-based thrombin generation assay.

15. The method of claim 14, wherein the endothelial cell-dependent activation of the protein C anticoagulant pathway is provided by introduction of a surrogate endothelial component.

16. The method of claim 15, wherein the surrogate endothelial component comprises quiescent EA.hy926 cells.

17. A method of assessing the effect of endothelial cell-dependent activation of the protein C anticoagulant pathway mediated by TM, EPCR, and/or TFPI on thrombin generation comprising:
a) providing plasma in the presence of endothelial cell-dependent activation of the protein C anticoagulant pathway; and
b) subjecting said plasma to a fluorogenic substrate-based thrombin generation assay.

18. The method of claim 17, wherein the endothelial cell-dependent protein C pathway is provided by introduction of a surrogate endothelial component.

19. The method of claim 18, wherein the surrogate endothelial component comprises quiescent EA.hy926 cells.

20. A method of assessing the effect of endothelial cell-dependent activation of the protein C anticoagulant pathway inactivation of factors Va and VIIIa on thrombin generation comprising:
a) providing plasma in the presence of endothelial cell-dependent activation of the protein C anticoagulant pathway; and
b) subjecting said plasma to a fluorogenic substrate-based thrombin generation assay.

21. The method of claim 20, wherein the endothelial cell-dependent activation of the protein C anticoagulant pathway is provided by introduction of a surrogate endothelial component.

22. The method of claim 21, wherein the surrogate endothelial component comprises quiescent EA.hy926 cells.

23. A method of testing plasma for a thrombin mediated condition or disease comprising:
a) providing plasma in the presence of endothelial cell-dependent activation of the protein C anticoagulant pathway; and
b) subjecting said plasma to a fluorogenic substrate-based thrombin generation assay.

24. The method of claim 23, wherein the endothelial cell-dependent activation of the protein C anticoagulant pathway is provided by introduction of a surrogate endothelial component.

25. The method of claim 24, wherein the surrogate endothelial component comprises quiescent EA.hy926 cells.
